(19) United States
(12) Patent Application Publication
(10) Pub. No.: US 2016/0287427 A1
(43) Pub. Date: Oct. 6, 2016

(54) GRAVITY-COMPENSATING ARM SUPPORT

(71) Applicant: The Nemours Foundation, Jacksonville, FL (US)

(72) Inventor: Tariq Rahman, Wilmington, DE (US)

(73) Assignee: The Nemours Foundation, Jacksonville, FL (US)

(21) Appl. No.: 15/083,534

(22) Filed: Mar. 29, 2016

Related U.S. Application Data
(60) Provisional application No. 62/141,544, filed on Apr. 1, 2015.

(51) Int. Cl. A61F 5/37; F16M 11/06

(52) U.S. Cl. CPC A61F 5/3738; F16M 11/06; F16M 2200/041

(57) ABSTRACT

A gravity-balance device, which may be an orthotic support, has a support, a lever pivotally mounted on the support, a spring extending from the support to the lever, and a cam mounted on one of the lever and the support. The cam is positioned so that the spring wraps round the cam as the lever pivots, altering the effective point of action of the spring. A thing being supported can hang from the lever. The thing may be a sling supporting a human arm parallel to the lever, so that the spring compensates for the weight of the arm as the arm is raised and lowered. The cam may be a smooth curve, a polygon, or a row of pins at the corners of an imaginary polygon.

### CROSS-REFERENCE TO RELATED APPLICATION

This application claims benefit of commonly invented and assigned U.S. provisional patent application No. 62/141,544 filed on 1 Apr. 2015 for a Gravity-Compensating Arm Support, the whole contents of which are incorporated by reference as if set forth explicitly herein.

### BACKGROUND

The present application relates generally to compensating for the force of gravity on a movable arm. One use of the present disclosure is in a passive overhead sling, supporting the arm of a human patient, to enable the patient to raise and move his or her arm, when the patient's own muscles are not sufficiently strong.

When an arm, human or mechanical, that is pivoted at a base or proximal end extends in a direction other than the vertical, a force or torque about the pivot must be exerted to support the arm against gravity. The torque required can be substantial, and varies depending on the angle of the arm to the horizontal. For example, when a typical 180 lb (80 kg) human adult holds his or her arm horizontal, the torque at the shoulder is around 9.5 ft-lbf (12.5 Nm).

The use of frictional force to hold the arm up is often undesirable, because moving the arm then requires an additional force to overcome the friction. The use of motors is often undesirable, because of both capital and operating cost and weight. For supporting the arm of a human patient, it is also difficult to control the motors so as to respond naturalistically to the patient's attempt to move the arm.

It has previously been proposed to use a spring arranged and positioned so that both the stretched length of the spring and its line of action relative to the pivot vary with the position of the arm. The effective torque exerted by the spring can then be matched to the gravitational torque on the arm. See, for example, Patent Specification No. GB 404,615 to George Carwardine, dated January 1934. However, the compensation provided is only approximate. These designs therefore still either require friction in the pivots to hold the arm steady, and an active force to overcome the friction and move the arm, or require an active force to hold the arm steady in a non-vertical position.

There are some circumstances in which the required active force is not readily available. For example, some patients with severe symptoms of stroke, spinal cord injury, muscular dystrophy, spinal muscular atrophy, and certain other neuromuscular conditions, have so little strength in their arms that they are unable to use a sling with Carwardine's geometry. In particular, during rehabilitation therapy, there may be a period when repeated movement of the arm is desirable to re-accustom the nervous system and muscles to movement, but the muscles are very weak, and easily fatigued. The lower the load on the muscles, the higher the number of repetitions of the exercise that is possible before fatigue sets in, and the more rapidly rehabilitation progresses. Consequently, a gravity balance support that balances the weight of the arm as exactly as possible is desirable.

The present inventor has previously shown that a single spring can in theory produce exact compensation for an arm pivoting in a vertical plane, if the spring has zero unstretched length, which is impossible for a real spring. See Rahman et al., A Simple Technique to Passively Gravity-Balance Articulated Mechanisms, Journal of Mechanical Design, Vol. 117, pp. 655-658, December 1995. In that paper it was shown that a spring with zero unstretched length can be simulated by a spring passing over a pulley, so that the unstretched length is outside the geometry of the gravity-balancing mechanism. However, when a spring, or an elastic cord, is passed over a pulley to simulate a spring of zero unstretched length, friction at the pulley can significantly impair the function of the mechanism in practice.

### SUMMARY OF THE INVENTION

Embodiments of the invention provide a gravity-balance device comprising a support, a lever or arm pivotally mounted on the support, a spring extending from the support to the lever, and a cam mounted on one of the lever and the support, wherein the cam is so positioned that the spring wraps round the cam as the lever pivots, altering the effective point of action of the spring.

The cam may be of any desired form. For example, it may have a convex curve around which the spring wraps, or it may have a polygonal shape around which the spring wraps. If the cam is polygonal, part or all of it may be in the form of pins, bars, or other structures supporting the spring at the corners of the polygon, with spaces in between.

The spring may be attached to the support above the pivot, and to the lever distally of the pivot, or the spring may be attached to the support below the pivot, and to an extension of the lever on the opposite side of the pivot from the operative part of the lever. There may be two springs, and they may then be positioned one above and one below the pivot.

Throughout this specification, words such as “above” and “below” are used to refer to an intended orientation of the mechanism in use to compensate for the force of gravity. However, the described devices may be made, transported, and stored in any orientation, and if used to compensate for a force other than gravity, may be used in an orientation appropriate to the force in question.

The spring may comprise a first portion and a second portion, where the first portion wraps around the cam and the second portion has a much lower elastic modulus than the first portion. Preferably, the elastic modulus of the second portion is so much lower than that of the first portion that stretching of the first portion is negligible for practical purposes. The second portion may comprise a coil of a coil spring, and the first portion may be an uncoiled length of the same material as the coil spring. Alternatively, the first portion may be of a different material from the second portion. The second portion may then be of natural or synthetic rubber or other elastomeric or similarly elastic material, such as bungee cord or rubber band.

The device may be an orthotic support, and the lever may then carry a sling for supporting an arm of a human patient. The geometry of the device, including the properties of the spring, may then be calculated to compensate correctly for the combined force of gravity on the device lever, the sling, and the patient's arm. The device may be optimized for a specific patient, or preset for a patient of a typical size.

### BRIEF DESCRIPTION OF THE DRAWINGS

The above and other aspects, features, and advantages of the present invention may be more apparent from the following more particular description of embodiments thereof, presented in conjunction with the following drawings. In the drawings:

FIG. 1 is a somewhat schematic side view of an overhead sling device for supporting the arms of a human patient.

FIG. 2 is a detail of FIG. 1, to a larger scale than FIG. 1.

FIG. 3 is an enlarged view of a cam in FIG. 2.

FIG. 4 is a diagram, similar to FIG. 3, of a gravity-balance device with a cam comprising four pegs.

FIG. 5 is a graph illustrating the performance of a gravity-balance device according to FIG. 4.

### DETAILED DESCRIPTION OF THE DRAWINGS

A better understanding of various features and advantages of the present methods and devices may be obtained by reference to the following detailed description of illustrative embodiments of the invention and accompanying drawings. Although these drawings depict embodiments of the contemplated methods and devices, they should not be construed as foreclosing alternative or equivalent embodiments apparent to those of ordinary skill in the subject art.

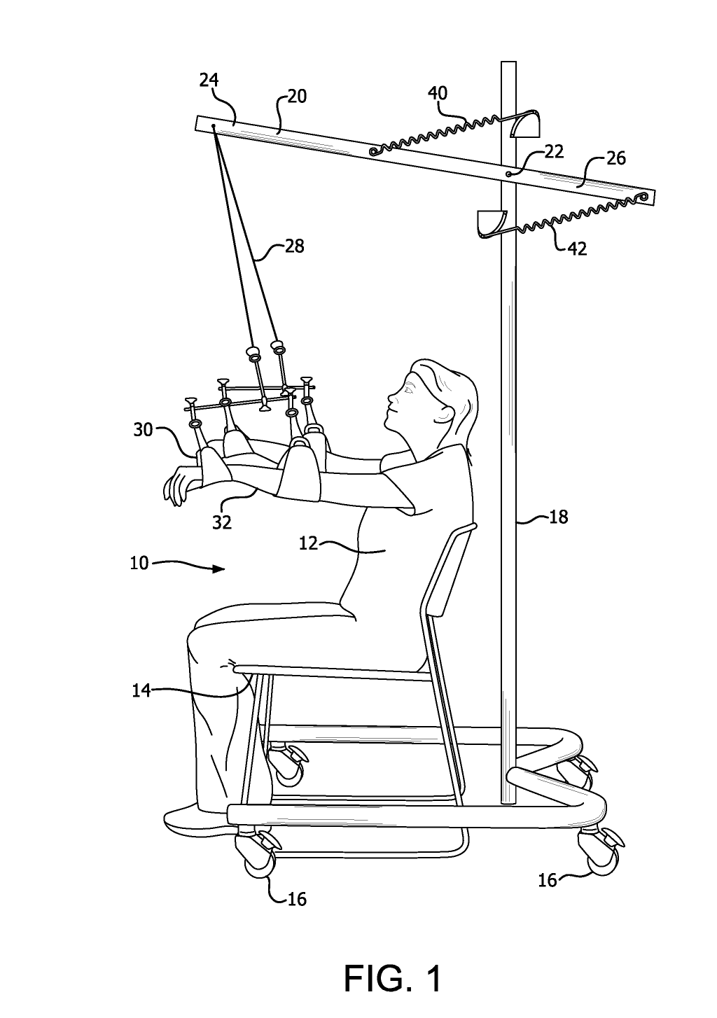

Referring to the drawings, one form of gravity-balance arm-support device suitable for use in rehabilitative therapy is indicated generally by the reference numeral 10. As shown in FIG. 1, a patient 12 sits on a chair 14. The device 10 is mounted on casters 16, and is positioned around the chair 14. The device 10 has a generally upright column 18, which in use is positioned behind the chair 14, and a lever 20 is attached by a pivot 22 near the top of column 18.

An operative end 24 of lever 20 extends forwards, over chair 14, and an extension 26 of lever 20 extends in the opposite direction, behind pivot 22. One or more ropes 28 hang from the operative end 24 of lever 20, and support a sling 30. The sling 30 supports the arm 32 of the patient 12. It may be seen from FIG. 1 that the simplest geometry is achieved when the pivot 22 is exactly above the shoulder joint of the patient 12, the arm 32 of the patient is parallel to the lever 20, and the ropes 28 are vertical. In practice, however, it is estimated that a device similar to that shown in FIG. 1, with the column 18 just behind the patient's shoulder, is adequate for up to 99% of the rehabilitation exercises for which the device 10 would be used.

In practice, device 10 may be provided with two columns 18, and two levers 20, spaced apart sideways and aligned to pass directly over the shoulders of patient 12, and each supporting one arm 32. In the interests of clarity, only one side will be described. The other may be substantially identical.

A pair of springs 40, 42 extend between column 18 and lever 20. Springs 40, 42 are coil springs acting in tension. One spring 40 is attached to column 18 above pivot 22, and to operative end 24 of lever 20 in front of pivot 22. The other spring 40 is attached to column 18 below pivot 22, and to extension 26 of lever 20 behind pivot 22. Thus, the linear forces exerted by springs 40, 42 tend to cancel out, thereby eliminating or at least reducing friction at the pivot 22, but each of the springs 40, 42 exerts a torque about pivot 22 that tends to raise the operative end 24 of lever 20, and the total torque from the two springs thus tends to support arm 32 against gravity.

As shown in more detail in FIG. 3, the end of spring 40 that attaches to column 18 has a bendable but not stretchy portion 44 that wraps over a cam 46 mounted on column 18, and is attached at 48 at the far end of cam 46. Steel wire is found to be a good material for the bendable portion 44. Steel wire has a high strength to weight ratio, high flexibility, and low lengthwise elasticity, and is widely available and economical.

Cam 46 has a surface that is convex upwards and forwards, so that the effective point of action of spring 40 on column 18 is the point 50 where bendable spring portion 44 meets cam 46 tangentially. Thus, as operative end 24 of lever 20 descends, bendable spring portion 44 wraps further round cam 46, and the line of action of spring 40 is lifted further from pivot 22. As is explained in more detail below, by suitable shaping of cam 46, it is possible for spring 40 to achieve a very accurate compensation for the torque exerted by the weight of lever 20, sling 30, and arm 32 as patient 10 raises and lowers arm 32.

As may be seen from the drawings, spring 42 is also provided with a bendable end portion 52 and cam 54. Those parts may be identically constructed, and symmetrically placed, to spring 40, bendable end 44, and cam 46.

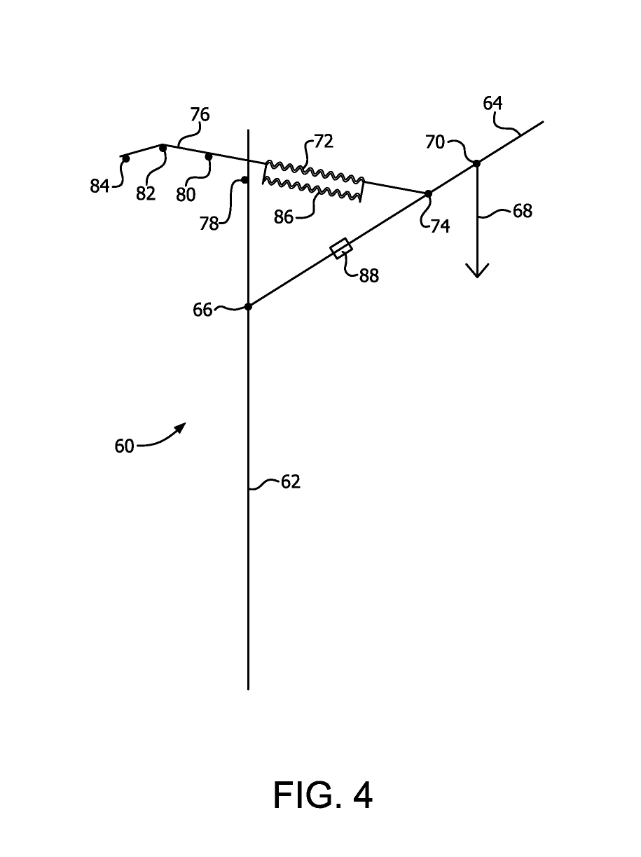

Referring now to FIG. 4, one embodiment of the device, indicated generally by the reference numeral 60, has a vertical column 62, to which a lever 64 is attached at a pivot 66. A sling 68 supporting the weight of a patient's arm (not shown) is attached to the lever 64 at a point 70. A spring 72 is attached to the lever 64 at a point 74, between points 66 and 70, and is attached to a wire rope 76, which passes over a cam consisting of four pins 78, 80, 82, 84. The nearest pin 78 is mounted on column 62 vertically above pivot 66. The other pins are mounted on a bracket (not shown in the interests of clarity) on the side of column 62 away from lever 64. The outer end of wire rope 76 is attached to the last pin 84, or to an anchor beyond and below pin 84.

As an example of how the embodiment shown in FIG. 4 may be implemented, the following derivations of equilibrium equations for the overhead sling arrangement may be applied. There are 4 stages as the wire wraps around each point. Unless otherwise stated, the subscript number 1 through 4 suffixed to a variable indicates the state of the variable in the respectively numbered stage.

Stage 1: arm 64 low, wire rope 76 resting on first pin 78, which is the pin nearest to the point 74 where the spring 72 attaches to arm 64 (which pin 78 is in this example vertically above the pivot 66).

$$sin {ξ}_{1} = \frac{{t}_{1}}{i} and \frac{b}{sin {ξ}_{1}} = \frac{{x}_{1}}{sin ( 180 - θ )}$$

### Where:

ξ is the angle, at the respective pin, between the direction to the pivot 66 and the direction to the point 74 where the spring 72 attaches to the arm 64;

t is the perpendicular distance from the pivot 66 to the line of the spring 72;

i is the vertical height from the pivot 66 to the pin 78;

b is the length of the arm 64 from the pivot 66 to the point 74;

x is the effective length of the spring; and

θ is the angle of the arm 64 to the vertically downward direction.

By rearranging those two equations to eliminate ξ₁:

$$⇒ \frac{bi}{{t}_{1}} = \frac{{x}_{1}}{sin θ} ⇒ {t}_{1} = \frac{bi sin θ}{{x}_{1}} and {x}_{1}^{2} = {i}^{2} + {b}^{2} - 2 ib cos ( 180 - θ ) ⇒ {x}_{1} = \sqrt{{i}^{2} + {b}^{2} + 2 ib cos θ}$$

The balance of forces on the arm 64 is

ΣM₀=mgl sin θ−k(x₁−x₀)t₁

where ΣM₀ is the net moment acting on the arm about the pivot 66;

m is the mass hanging from the point 70, including an allowance for the weight of the arm 64;

l is the distance from the point 70 to the pivot 66;

k is the spring rate of the spring 72, expressed as force per unit increase in length; and

x₀ is the unstretched length of the spring 72, which in this calculation is assumed in stage 1 to occupy the entire space between the attachment point 74 and the first pin 78.

The net moment ΣM₀ is equal (but of opposite sign) to the moment that must be exerted by the patient's muscles or some other external force to keep the arm in equilibrium. For many of the proposed uses of this embodiment, ΣM₀ should ideally be equal to zero at all values of θ. In a real embodiment, that ideal is not exactly possible but, as will be shown below, an approximation can be achieved that is sufficiently close to be good for most therapeutic uses.

Stage 1 applies over the range of angles θ>0 to:

$$θ < \{ {sin}^{- 1} [ \frac{in}{b \sqrt{{( j - i )}^{2} + {n}^{2}}} ] + {sin}^{- 1} [ \frac{n}{\sqrt{{( j - i )}^{2} + {n}^{2}}} ] \} \qquad [ 1 ]$$

Stage 2: wire rope 76 resting on second pin 80, but not on first pin 78.

$$sin {ξ}_{2} = \frac{{t}_{2}}{\sqrt{{j}^{2} + {n}^{2}}} and \frac{b}{sin {ξ}_{2}} = \frac{{x}_{2}}{sin ( 180 - θ + {β}_{2} )}$$

where:

j is the vertical distance from pivot 66 to pin 80;

n is the horizontal distance from pivot 66 to pin 80; and

β is the angle at the pivot 66 between a vertically upward direction and the direction to the pin 80. (β₁ was not mentioned because it is zero.)

From the above equation it follows that:

$$⇒ {t}_{2} = \frac{\sqrt{{n}^{2} + {j}^{2}} b sin ( θ - {β}_{2} )}{{x}_{2}}$$

$$sin {β}_{2} = \frac{n}{\sqrt{{n}^{2} + {j}^{2}}} and {x}_{2}^{2} = {b}^{2} + {n}^{2} + {j}^{2} - 2 b \sqrt{{n}^{2} + {j}^{2} cos ( 180 - θ + {β}_{2} )} \\ ⇒ {x}_{2} = \sqrt{{b}^{2} + {n}^{2} + {j}^{2} + 2 b \sqrt{{n}^{2} + {j}^{2}} cos ( θ - {β}_{2} )}$$

$$Σ {M}_{0} = mgl sin θ - k ( {x}_{2} - {x}_{0} - \sqrt{{( j - i )}^{2} + {n}^{2}} ) {t}_{2}$$

Stage 2 applies over the range of angles:

$$\leq θ < \{ {sin}^{- 1} [ \frac{\sqrt{{j}^{2} + {n}^{2}} sin {ξ}_{2}}{b} ] + 90 - α \} \\ where α = {sin}^{- 1} [ \frac{f - j}{\sqrt{{( f - j )}^{2} + {( d - n )}^{2}}} ] \\ {ξ}_{2} = 90 - {β}_{2} - α \\ {β}_{2} = {sin}^{- 1} [ \frac{n}{\sqrt{{j}^{2} + {n}^{2}}} ] \qquad [ 1 ]$$

Stage 3: wire rope 76 resting on third pin 82, but not on second pin 80:

$$sin {ξ}_{3} = \frac{{t}_{3}}{\sqrt{{d}^{2} + {f}^{2}}} and \frac{b}{sin {ξ}_{3}} = \frac{{x}_{3}}{sin ( 180 - θ + {β}_{3} )}$$

where:

f is the vertical distance from pivot 66 to pin 82; and

d is the horizontal distance from pivot 66 to pin 82.

$$⇒ {t}_{3} = \frac{\sqrt{{d}^{2} + {f}^{2}} b sin ( θ - {β}_{3} )}{3}$$

$${x}_{3}^{2} = {b}^{2} + {d}^{2} + {f}^{2} - 2 b \sqrt{{d}^{2} + {f}^{2}} cos ( 180 - θ + {β}_{3} ) \\ ⇒ {x}_{3} = {b}^{2} + {d}^{2} + {f}^{2} + 2 b \sqrt{{d}^{2} + {f}^{2}} cos ( θ - {β}_{3} )$$

$$Σ {M}_{0} = mgl sin θ - k ( {x}_{3} - {x}_{0} - \sqrt{{( j - i )}^{2} + {n}^{2}} - \sqrt{{( f - j )}^{2} + {( d - n )}^{2}} ) {t}_{3}$$

The range of angles θ for Stage 3 may be determined as follows:

180−θ+β₄+φ₄+ξ₄=180

where: φ₄ is the angle at the point 74 between the direction to the pivot point 66 and the direction to the fourth pin 84 (which at the limit between stages 3 and 4 is also the direction to the third pin 82).

From this it follows that:

$$⇒ θ = {β}_{4} + {∅}_{4} + {ξ}_{4} = {β}_{4} + {∅}_{4} + ( 90 - {β}_{4} + {sin}^{- 1} [ \frac{f - p}{\sqrt{{( f - p )}^{2} + {q}^{2}}} ] )$$

where:

p is the vertical distance from pivot 66 to pin 84; and

q is the horizontal distance from pin 82 to the fourth pin 84.

That can be simplified to:

$$θ = {∅}_{4} + 90 + {sin}^{- 1} ( \frac{f - p}{\sqrt{{( f - p )}^{2} + {q}^{2}}} )$$

But

$$\frac{b}{sin {ξ}_{4}} = \frac{\sqrt{{( d + q )}^{2} + {p}^{2}}}{sin {∅}_{4}} ⇒ sin {∅}_{4} = \frac{sin {ξ}_{4} \sqrt{{( d + q )}^{2} + {p}^{2}}}{b}$$

Therefore,

$$θ = {sin}^{- 1} [ \frac{sin {ξ}_{4} \sqrt{{( d + q )}^{2} + {p}^{2}}}{b} ] + {β}_{4} + {ξ}_{4} \qquad [ 2 ]$$

where

$${β}_{4} = {sin}^{- 1} [ \frac{d + q}{\sqrt{{( d + q )}^{2} + {p}^{2}}} ]$$

$${ξ}_{4} = 90 - {β}_{4} + {sin}^{- 1} [ \frac{f - p}{\sqrt{{( f - p )}^{2} + {q}^{2}}} ]$$

Therefore, the range of values of 0 for stage 3 is:

$${sin}^{- 1} [ \frac{\sqrt{( {j}^{2} + {n}^{2} ) sin {ξ}_{2}}}{b} ] + 90 - α \leq θ < [ 2 ]$$

Stage 4: wire rope 76 coming directly from fourth, rearmost pin 84:

$$sin {∅}_{4} = \frac{{t}_{4}}{b} and \frac{\sqrt{{( d + q )}^{2} + {p}^{2}}}{sin {∅}_{4}} = \frac{{x}_{4}}{sin ( 180 - θ + {β}_{4} )} ⇒ \frac{b \sqrt{{( d + q )}^{2} + {p}^{2}}}{{t}_{4}} = \frac{{x}_{4}}{sin ( θ - {β}_{4} )} ⇒ {t}_{4} = \frac{b \sqrt{{( d + q )}^{2} + {p}^{2}} sin ( θ - {β}_{4} )}{{x}_{4}}$$

### and ΣM₀=mgl sin θ

−k(x₄−x₀−\sqrt{(j−i)²+n²}−\sqrt{(f−j)²+(d−n)²}−\sqrt{(f−p)²+q²})t₄

The range of values of θ for Stage 4 is:

180°≧θ≧[2]

As an example of suitable dimensions, taking the pivot 66 as the origin of Cartesian coordinates, with +X horizontally towards the free end of lever 64 and +Y vertically upwards:

The length of lever 64 between points 66 and 74 is 8″ (20 cm);

The length of lever 64 between points 66 and 70 is 10″ (25 cm);

The four pins 78, 80, 82, 84 are at coordinates (0, i)=(0, 2.9), (n, j)=(0.25, 3.2), (d, f)=(1.4, 3.5), and (d+q, p)=(2.1, 3.4), where the dimensions are in inches, and taking the pivot 66 as the origin of Cartesian coordinates, with +X horizontally away from the sling 68 and +Y vertically upwards.

However, as noted above, the value of X₀ used for the calculations includes the part of the wire rope 76 that in stage 1 is to the right of the first pin 78, and any inelastic attachments between the coiled part and the point 74. The spring rate K of spring 72 is 1.9 lbf-in.

When the spring 72 is in its unstretched, but not slack, position, the lever 64 is vertically upwards (at an angle of 90 degrees above the horizontal, θ=180°), and the total straight length of spring 72 and wire rope 76 from point 74 to pin 84 is 5.06 inches. The unstretched length X₀ of the actual elastic part of spring 72 is 2.8 inches. The length of the inelastic wire rope from point 74 to the beginning of the elastic part 72 is then 2.26 inches. That results in the joint or changeover between the inelastic and elastic parts being at the pin 84 in stage 1, which is conceptually the simplest arrangement.

Those dimensions are believed to provide a device 10 that will be suitable for use with most otherwise normal humans. The rate of the spring may be chosen as follows, based on the patient's body weight:

| Body weight, lbf | Spring stiffness, lbf-in | weight of arm, lbf |
|---|---|---|
| 50 | 1.9 | 3.15 |
| 100 | 3.5 | 5.80 |
| 150 | 5.1 | 8.45 |
| 200 | 6.8 | 11.10 |

where “weight of arm” is the weight of the patient's arm plus 0.5 lbf for the weight of the lever 64 and sling 68.

In a practical embodiment, the rate of the spring 72 may be set by changing the spring, or by adding and removing additional springs 86 in parallel. Alternatively, the spring 72 may be set for the heaviest patient, and the device 60 may be adjusted by a rider weight 88 that is added, removed, or slid along the arm 64, so that the combined gravitational moment (weight multiplied by distance from pivot 66) of the actual patient's arm and the rider weight 88 equals the moment for which the spring 72 is calculated. A rider weight would increase the angular momentum of the lever 64, so that the device 60 would respond less quickly to the patient's movements. Depending on the exact physical therapy exercise being performed, the increased angular momentum may be desired, undesired, or unimportant.

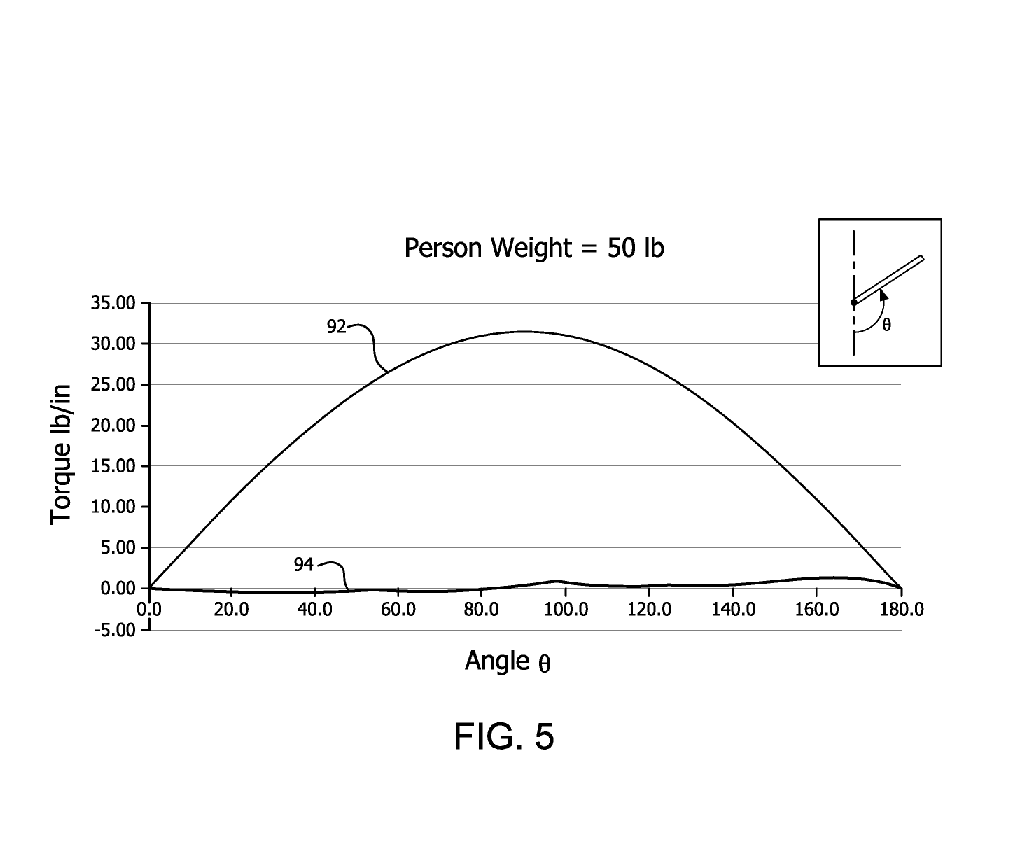

FIG. 5 is a graph showing the calculated torque at the shoulder required to support the weight of a fully extended human arm over a range of angles from θ=0 (vertically downwards) to θ=180° (vertically upwards) without additional support, curve 92, and when supported by the device shown in FIG. 4 correctly adjusted, curve 94. For a 50 lb person, the ordinate extends from 0 to 35 lbf-in. As may be seen in FIG. 5, without additional support, the maximum torque (with the arm horizontal) is about 32 lbf-in. Using the device 60 for support, the torque never exceeds 2 lbf-in. For a heavier person, with correct adjustment to the springs 72, 86 or the rider weight 88, both maximum torques would increase approximately in proportion to the weight of the patient.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

For example, the device 10 shown in FIG. 1 has the lever 20 high above the patient 12. That arrangement has the advantage of being easy to understand in the drawing. However, the lever 10 could be directly combined with the sling 30, or positioned at any other convenient height.

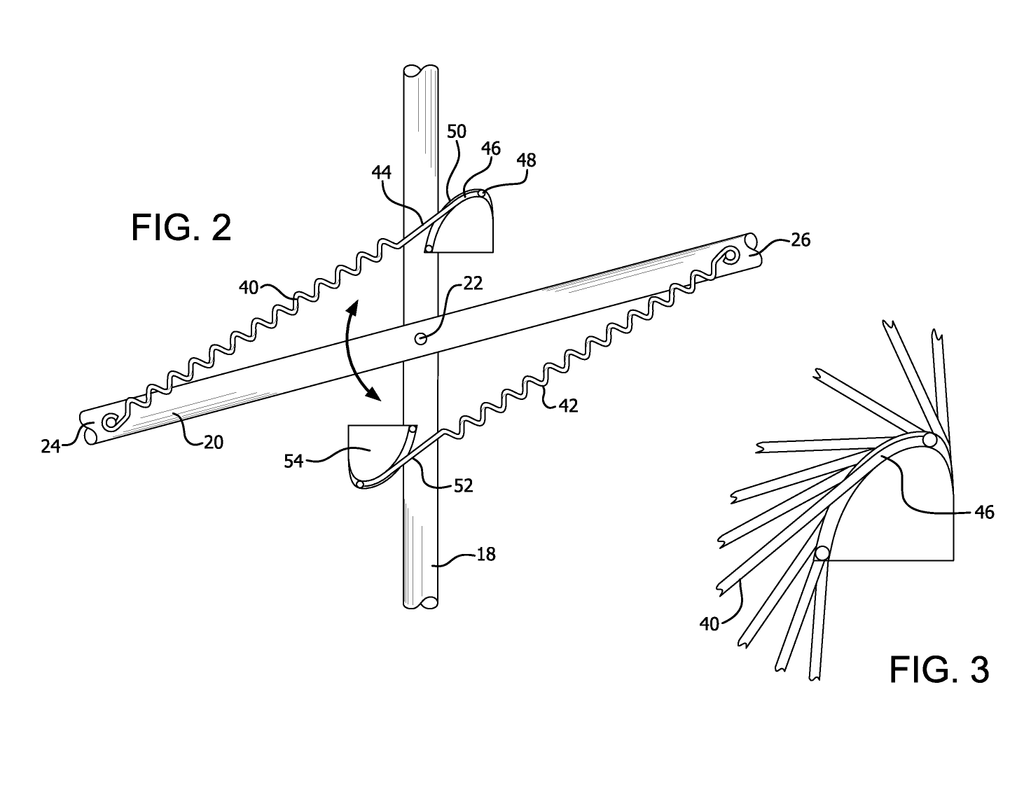

Although two symmetrically disposed springs 40, 42 are shown in FIG. 2, either one of those springs alone could be used, provided that the strength of the spring is doubled. Conversely, although only one spring 72 is shown in FIG. 4, two springs 72 arranged similarly to springs 40, 42 in FIG. 2 could be used. Alternatively, two or more identical or non-identical springs could be provided on one side of the pivot 22, for example, if it was desirable to eliminate lever extension 26 or the part of column 18 above pivot 22. In most cases, a single spring and cam are preferred for simplicity and ease of manufacture. Also, in embodiments with an adjustable or interchangeable spring 72, 86, it is easier for the operator to have only one spring to adjust. However, an arrangement with two balanced springs reduces the reaction force, and therefore the friction, at the pivot 66, and may be preferred where smooth operation is important.

Although cams 46, 54 are shown in FIGS. 2 and 3 with smoothly curved surfaces, other arrangements are possible. For example, the cam surfaces could be polygonal, which would then be geometrically equivalent to the row of pins 78, 80, 82, 84. In FIG. 4, the number of pins could be more or fewer than four. A smaller number of pins might allow for easier construction, but a larger number of pins, towards the limiting case of a continuous curve, is usually preferred because it allows for smoother motion and greater precision in the gravity compensation.

In order to determine the optimum dimensions for a different configuration, Applicant has found that the most efficient approach is to set up the key equations in a spreadsheet program, and adjust the independent variables iteratively until a sufficiently good solution is found. The skilled person will easily understand from the given example how to set up the equations for a configuration other than the four pins used in the example. If reasonable starting estimates are chosen, and reasonable adjustments are made at each iteration, the equations should converge quite quickly. Alternatively, or for more complex configurations such as a number of pins much larger than four, a computer program using a standard hill-climbing algorithm can be used.

In the interests of clarity and simplicity, the springs 40, 42, 72 are symbolized in the drawings as coil springs, with in some instances an uncoiled end. As noted above, in the example calculation, it is assumed that the elastic length of the spring 72 occupies the entire distance from the point where in stage 1 the wire rope 76 and spring 72 rest on the first pin 78, to the attachment point 74. The difference does not affect the calculation, provided that X₀ is correctly identified.

Instead of the illustrated coil springs, any appropriate device that acts as a tension spring may be used, for example, elastic cords or straps. Such cords or straps may be made of natural or synthetic rubber, other elastomers, or similarly elastic materials, such as bungee cords, rubber bands, or the, or the rubber strips of calibrated stiffness that are extensively used in physical therapy exercises. Differences in the spring rate of the spring may be compensated by positioning the spring nearer to or further from the pivot 22 or 66, and/or by changing how much of the assembly 40, 44 or 72, 76 is the actual spring and how much is a less stretchable tether. For example, FIG. 4 shows a straight section between spring 72 and attachment point 74 that could be either spring or wire rope.

Accordingly, reference should be made to the appended claims, rather than to the foregoing specification, as indicating the scope of the invention.

1. A gravity-balance device comprising:
a support;
a lever pivotally mounted on the support;
a spring extending from the support to the lever; and
a cam mounted on one of the lever and the support;
wherein the cam is so positioned that the spring wraps round the cam as the lever pivots, altering the effective point of action of the spring.

2. The device of claim 1, wherein the spring is attached to the support above the pivot, and to the lever distally of the pivot.

3. The device of claim 1, wherein the spring is attached to the support below the pivot, and to an extension of the lever on an opposite side of the pivot from an operative part of the lever.

4. The device of claim 1, comprising two said springs, wherein a first said spring is attached to the support above the pivot, and to the lever distally of the pivot, and a second said spring is attached to the support below the pivot, and to an extension of the lever on an opposite side of the pivot from an operative part of the lever.

5. The device of claim 1, wherein the spring comprises a first portion and a second portion, wherein the first portion wraps around the cam and the second portion has a lower elastic modulus than the first portion.

6. The device of claim 5, wherein the spring second portion comprises a coil of a coil spring, and the spring first portion is an uncoiled length of a same material as the coil spring.

7. The device of claim 5, wherein the second spring portion is selected from the group consisting of natural and synthetic rubber and other elastomeric materials.

8. The device of claim 1, wherein the cam comprises a three or more discrete supports arranged in a curved line.

9. The device of claim 1, which is an orthotic support, and wherein the lever carries a sling for supporting an arm of a human patient.
